(19) United States
(12) Patent Application Publication
(10) Pub. No.: US 2020/0100918 A1
(43) Pub. Date: Apr. 2, 2020

(54) Garment with loop and pocket for breast prostheses

(71) Applicant: Nicole Elisabeth Marie Mommers-McCaffrey, New Milford, CT (US)

(72) Inventor: Nicole Elisabeth Marie Mommers-McCaffrey, New Milford, CT (US)

(21) Appl. No.: 16/700,539

(22) Filed: Dec. 2, 2019

Related U.S. Application Data
(63) Continuation of application No. 15/988,324, filed on May 24, 2018.

(51) Int. Cl. A61F 2/52; A41B 1/08; A41D 1/22; A41C 3/14

(52) U.S. Cl. CPC A61F 2/52; A41C 3/144; A41D 1/22; A41B 1/08

(57) ABSTRACT

A garment containing a pocket and loop to securely hold a single length of padding with bilateral breast mound prostheses for the mastectomy patient or other is disclosed. The garment includes a front layer, a middle section with a loop and a back layer. The single length of padding with bilateral breast mound prostheses is securely placed into the pocket and loop component of the garment. The garment is also able to take on various forms in order to securely hold two separate breast mound prostheses if desired.

### CROSS-REFERENCES

This application claims the benefit of U.S. Provisional Patent Application No. 62/774,428 filed on Dec. 3, 2018 and U.S. Non-Provisional patent application Ser. No. 15/988,324 filed on May 24, 2018 which are hereby incorporated by reference in their entirety.

### BACKGROUND OF THE INVENTION

### Field of the Invention

This garment present invention relates to mastectomy clothing and breast prosthetics that are placed inside such mastectomy clothing. More specifically, this garment invention relates to a convenient and comfortable way to dress with breast prosthetics without the use of broadband elastic within the garment. The single length of bilateral breast mound prosthetics are intended to be secured through the loop inside the chest pocket of the garment, thus offering a comfortable, convenient and confident way to dress with mastectomy clothing and breast prosthetics without the use of broadband elastic.

### Background

Research shows that many women have a psychological need for breast replacement after mastectomy whether it is permanent or temporary. There are few options available today that are temporary, comfortable, non-constricting and convenient. Women are typically offered one or more breast prosthetics that are placed inside a garment with broadband elastic. These garments can be constrictive, heavy, uncomfortable, unattractive and inconvenient. This invention comes from the need and desire of wanting to restore a temporary and aesthetically-correct breast mound appearance comfortably, safely and conveniently after mastectomy surgery while also wanting to protect the sensitive pectoral and sternal areas caused by mastectomy. It is important for the mastectomy patient to not wear any constricting garments because of the increased risk of Lymphedema after mastectomy and lymph node removal.

Lymphedema is a condition of localized fluid retention in the body which can be worsened by wearing ill-fitting elastic-banded bras or garments. This garment invention rids the risk of any constriction when the breast mound prosthetics component is secured into the garment invention appropriately and intentionally because there is no broadband elastic and no tightness against the skin. There are mastectomy shirts on the market today but most are offered with elastic-banded pockets for a breast prosthesis and they can be very constrictive and uncomfortable against the mastectomy site. This garment present invention intends to solve the problem of uncomfortable mastectomy wear bu having no broadband elastic within the garment all while intending to help restore the self-confidence and mental well-being of a mastectomy patient or other person, in a safe, quick, lightweight and comfortable fashion.

### SUMMARY OF THE INVENTION

This garment present invention has resolved the above drawbacks of mastectomy wear on the current market by providing a comfortable, convenient and safe alternative to the many typical mastectomy garments and breast prostheses that are offered today. This garment present invention is intended to offer the benefit of this unique design in that it will hold one or more breast mound prostheses in a comfortable, convenient and safe fashion because it does not contain any broadband elastic. The garment present invention simply contains one or more front pockets and a loop which provide options to hold lightweight breast mound prosthetics. There is no elastic around the torso and no elastic under the breast line. This garment present invention is available in many different fashion designs but the integrity and essence of the garment remain in that it offers a quick, comfortable and safe mastectomy garment without the use of any broadband elastic.

### BRIEF DESCRIPTION OF THE DRAWINGS

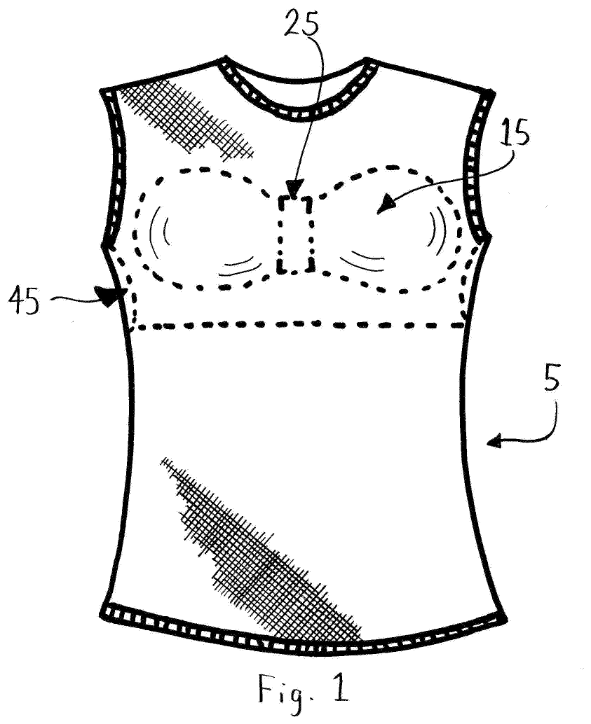

1. FIG. 1 illustrates a front view of one representation of the garment present invention.

2. FIG. 2 illustrates a front view of the one representation of the back layer of the garment present invention thereof;

3. FIG. 3 illustrates a front view of one representation of one part of the middle layer of the garment present invention thereof;

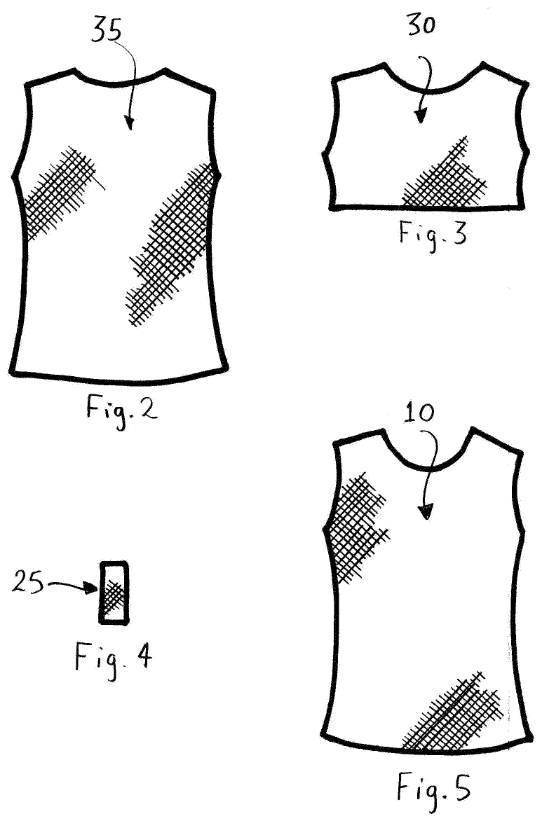

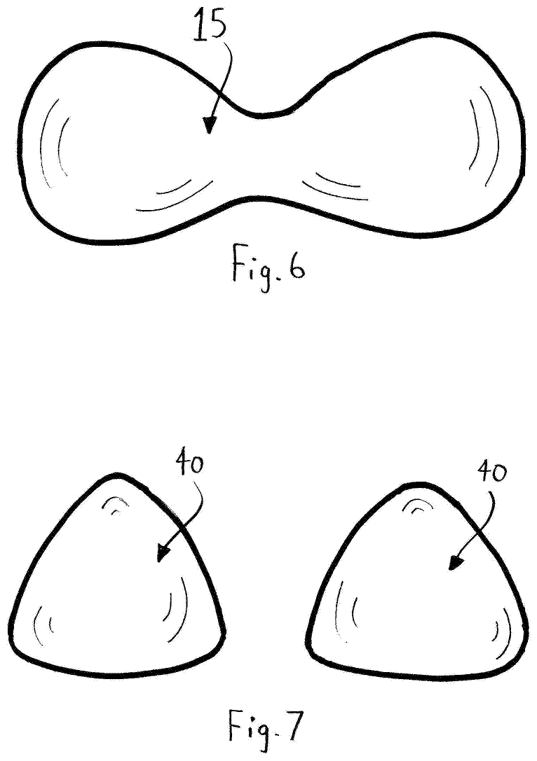

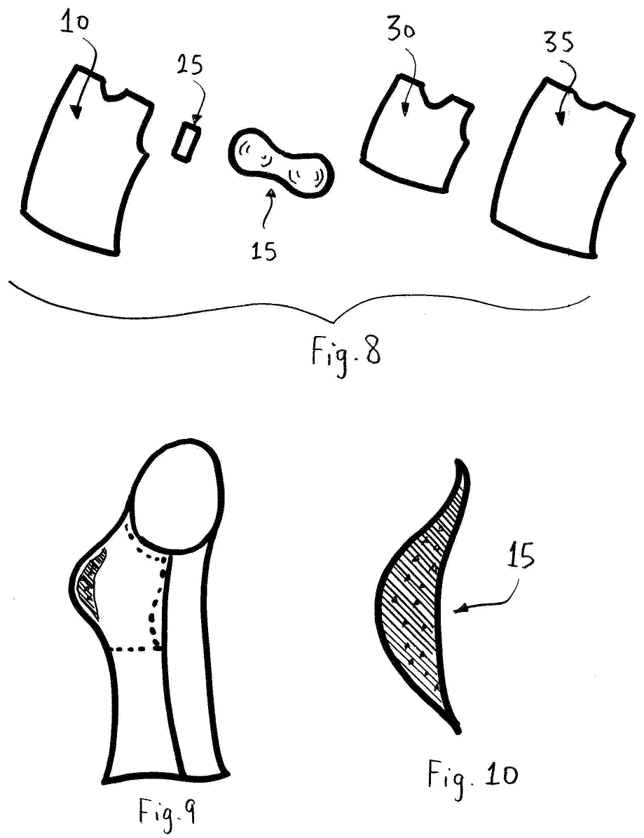

4. FIG. 4 illustrates a front view of one representation of the loop component of the garment present invention thereof;

5. FIG. 5 illustrates a front view of one representation of the front layer of the garment present invention thereof;

6. FIG. 6 illustrates a front view of one representation of a single length of padding with bilateral breast mound prostheses thereof;

7. FIG. 7 illustrates a front view of one representation of a two, single breast mound prostheses thereof;

8. FIG. 8 illustrates a front perspective, exploded view of one representation of the garment present invention thereof;

9. FIG. 9 illustrates a side elevation view of one representation of the garment present invention, the opposite being a mirror image thereof;

10. FIG. 10 illustrates a cross-sectional view of one representation of the breast mound prosthetic component of the garment present invention thereof;

11. FIG. 13 illustrates a front view of one representation of the garment present invention with a single length of padding with bilateral breast mound prostheses within the loop of the garment present invention thereof; and

12. FIG. 14 illustrates a front view of one representation of the garment present invention with two separate breast mound prostheses secured within the breast pockets of the garment present invention.

### DETAILED DESCRIPTION OF THE INVENTION

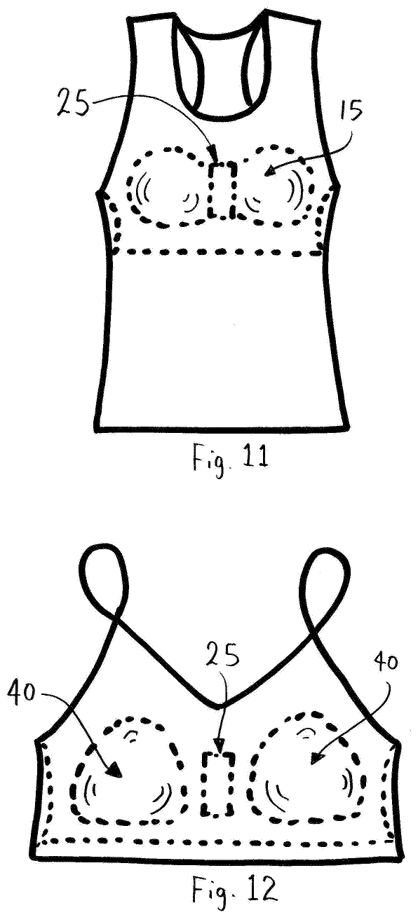

While this garment is invariable in its nature and essence that of being a garment with no broadband elastic and containing one or more pockets and a loop to offer the mastectomy patient, or other, options of wearing lightweight breast mound prosthetics in a comfortable fashion, this said garment invention and all its components is able to take on different shapes and sizes in order to properly accommodate any and all individuals wearing this garment present invention. This garment present invention includes a front layer of the garment, a middle section of the garment which may consist of one or more layers and a loop, a breast prosthetic or breast prostheses which are separate and optional, and a back layer of the garment unless it is a backless design of one representation of the garment present invention. One representation of various styles of the garment present invention 5 is illustrated in FIG. 1, FIG. 11 and FIG. 12. The front layer 10 of the garment present invention can be of any style of garment. The middle components of the garment can include the loop 25 a single middle layer 30 or more middle layers. The said components are attached to each other thus creating one or more pockets intended to house and hold a single length of bilateral breast mound prostheses 15 or separate breast mound prostheses 40. When a middle section of the garment is sewn vertically down the center of the said middle section it creates two pockets to accept two separate breast mound prostheses 40. When the middle section has no stitching vertically down the center but has a loop within the middle section this representation creates the pocket with loop intended to house and hold the single length of bilateral breast mound prostheses 15. The front layer 10, the loop 25, the middle layer 30 and the back layer 35 are all attached to each other to create one representation of the garment present invention 5. While a full and complete description of the garment present invention 5 has been announced in accordance with the patent statues it should be acknowledged that modifications can be made to any part of the garment present invention 5 without deviating from the essence and spirit of the claims.

What is claimed is:

1. A garment containing one or more pockets which intends to securely house and hold a single length of enhanced and contoured bilateral breast mound prostheses or two separate breast mound prostheses for the mastectomy patient or other consisting of:
a front layer of the garment which rests over the chest area
a middle section of the garment which rests over the chest area and a
a back layer of the garment which rests over the back of the body, all of which are joined together appropriately to form the garment present invention.

2. The garment according to claim 1, wherein the garment has no broadband elastic within the garment.

3. The garment according to claim 1, wherein the front layer, the middle section and the back layer may be of any size or design.

4. The garment according to claim 1, wherein the middle section of the garment has a loop of material that is attached to the said middle section of the garment in order to securely hold a single length of bilateral breast mound prostheses.

5. The garment according to claim 1, wherein the loop may not be included in the design in order to accommodate various styles of the garment present invention which can hold two separate breast mound prostheses.

6. The garment according to claim 1, wherein the middle section of the garment may be sewn to the front layer of the garment to form a pocket for the breast mound prostheses.

7. The garment according to claim 1, wherein the middle section of the garment may hang loose within the garment in order to accommodate various designs of the garment present invention.

8. The garment according to claim 1, wherein the middle section may have more than one layer in order to accommodate various styles of the garment present invention.

9. The garment according to claim 1, wherein the layers forming the pocket(s) may be sewn to the inside or outside of the front layer of the garment present invention.

10. The garment according to claim 1, wherein the garment may have additional stitching down the center of the middle section in order to accommodate various styles of the garment present invention which can securely hold two separate breast mound prostheses.

11. The garment according to claim 1, wherein the garment is intended to house and securely hold one or more lightweight breast prosthetics of any length, style or design.

12. The garment according to claim 1, wherein the breast prosthetics may be a single length of bilateral breast mound prostheses or two separate breast mound prostheses.

13. The garment according to claim 1, wherein the garment present invention is a garment with one or more elastic-free front pockets for breast prostheses.

14. The garment according to claim 1, wherein the breast prosthetics component of the garment invention are removable and separate from the garment present invention.
